(19) United States
(12) Patent Application Publication
(10) Pub. No.: US 2006/0051357 A1
(43) Pub. Date: Mar. 9, 2006

(54) Tolerance induction with help of a combination of a lfa-1 inhibitor and a costimulation inhibitor and or a mtor inhibitor

(76) Inventors: Andreas Katopodis, Oberwil (CH); Philip Lake, Morris Plains, NJ (US); Barbara Metzler, Lorrach (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/522,823

(22) PCT Filed: Jul. 30, 2003
(86) PCT No.: PCT/EP03/08436

§ 371 Date: Jul. 8, 2005

(30) Foreign Application Priority Data
Jul. 31, 2002 (GB) 0217777.2

(51) Int. Cl. A61K 39/00

(52) U.S. Cl. 424/184.1

(57) ABSTRACT

A method for inducing or modulating T or B cells tolerance to donor cells, tissue or organ in a recipient comprising administering to the recipient a LFA-1 inhibitor in combination with a costimulation inhibitor and/or a mTOR inhibitor.

The present invention relates to immune modulation for the induction of hematopoietic chimerism as a strategy for transplantation tolerance, autoimmune diseases, genetic deficiencies and cancers.

Over the last two decades, organ transplantation has become a routine therapeutic option for patients with end-stage organ failure. Both short-term and long-term outcomes after organ transplantation have improved; nevertheless, long-term morbidity and mortality still remain substantial problems. The chronic immunosuppression that organ transplant recipients require for the rest of their lives frequently fails to prevent graft loss due to chronic rejection and is associated with severe side-effects, including infections, malignancies, nephrotoxicity and metabolic disorders. The induction of immunological tolerance could provide a solution to these pressing problems not only in the field of allotransplantation but also in certain autoimmune diseases.

By immunological tolerance is meant specific lack of productive immunity in vivo against selected antigens. Immunological tolerance to allo-antigens therefore prevents graft rejection while maintaining the recipient's normal immune responsiveness to infections or other immunological stimuli.

Chimerism describes the establishment of donor-derived cells within a host. For many experimental and clinical purposes, chimerism induction is sought for hematopoietic cells and involves transplantation of donor bone marrow or bone marrow-derived stem cells. Depending on host pre-conditioning, this may develop as mixed chimerism with cells of both donor and host type co-existing within the host, or as full chimerism where donor-type cells replace a set of host cells. For the purpose of this invention chimerism refers to macro-chimerism defined as greater than or equal to 5% of donor-derived cells within one or more blood cell-compartment, e.g. granulocytes, lymphocytes.

A major-application of hematopoietic chimerism is the induction of central thymic tolerance to allo-antigens. Central immunological tolerance describes the process by which developing T cells with high affinity for self antigen are deleted in the thymus. In the unmanipulated immune system central tolerance is restricted to self antigens. In a chimeric hematopoietic system, antigen presenting cells of donor origin populate the host thymus and present a large repertoire of allo-antigens to T cell precursors of both host and donor origin. Consequently, allo-antigens may be interpreted and tolerated as neo-self antigens. This is the most stable form of immunological tolerance possible. In the light of problems and side effects still experienced with the use of relatively non-specific immunosuppressive agents, central tolerance through the induction of hematopoietic chimerism is considered a highly desirable goal for therapeutic intervention.

Mixed or full hematopoietic chimerism may also be therapeutically useful in the treatment of hematopoietic and other malignancies as well as hemoglobinopathies and immune deficiencies.

In order to establish hematopoietic chimerism, the recipient requires pre-treatment to 1. enable engraftment of donor hematopoietic precursor cells against resistance by homeostatic regulation of the recipient's precursor cell pools and 2. protect donor hematopoietic precursors from the recipients immune response until central tolerance is established.

A common method to reduce the recipient's own precursor cell numbers is by using myelosuppressive agents such as busulfan, fludarabine, etc, or irradiation. These treatments are hazardous, and a method to achieve hematopoietic chimerism with strongly reduced dosage or even without the use of such agents would be of great benefit to the patient.

In order to protect donor hematopoietic cells until central tolerance is achieved, short-term immune modulation of the recipient is required to prevent a productive allo-immune response against donor cells.

This kind of immune modulation may exploit insight into physiological immune strategies for ‘back-up’ tolerance in the periphery to prevent productive immunity against self antigens upon incomplete thymic deletion and altered patterns of self-recognition. One hallmark of peripheral tolerance is the lack of certain costimulatory receptor/ligand pairs formed between T cells and APC during cognate antigen-mediated interactions. The formation of costimulatory ‘cross-talk’ between APC and T cells is mandatory for productive immunity. Costimulation blockade in vivo, therefore, has often been the strategy of choice for experimental induction and/or maintenance of peripheral tolerance. Thus far, major costimulatory pathways blocked for the induction of chimerism have been the CD40/CD40L and CD28/CD80/CD86 ligand receptor pairs. In other experimental systems, blockade of ICOS, OX-40, and 4-1 BB (among others) has also been proposed.

It has now surprisingly been found a method for inducing stable, multi-lineage hematopoietic chimerism through inhibition of LFA-1 in vivo.

In accordance with the particular finding of the present invention, there is provided:
1.1 A method for inducing or modulating T or B cell tolerance to donor cells, tissue or organ in a recipient comprising administering to the recipient a LFA-1 inhibitor in combination with a costimulation inhibitor and/or a mTOR inhibitor.
1.2 A method for inducing hematopoietic chimerism in a recipient of cells, tissue or organ transplant from a donor comprising administering to the recipient
   i) bone marrow cells or other precursor cells from the donor; and
   ii) a LFA-1 inhibitor in combination with at least one co-agent selected from a co-stimulation inhibitor and a mTOR inhibitor.

As used herein, the term “transplant” refers to organs and/or tissues and/or cells which can be obtained from a first mammal (or donor) and transplanted into a second mammal (a recipient), preferably a human. The term “transplant” as well as “cells, tissue or organ” encompasses, for example, skin, eye or portions of the eye (e.g., cornea, retina, lens), bone marrow, muscle, heart, lung, heartlung, liver, kidney, pancreas (e.g., islet cells, β-cells), parathyroid, bowel (e.g., colon, small intestine, duodenum), neuronal tissue, bone and vasculature (e.g., artery, vein).

Precursor cells include e.g. stem cells, hematopoietic precursor cells or lymphoid cells and can be administered either as a mixture of several cell types as harvested from a donor or after purification and/or culturing to obtain an enriched or purified population.

The “transplant” may be transplanted in the recipient either concurrently with the bone marrow or precursor cells i) or at a time later after hematopoietic chimerism has been first established in the recipient.
1.3 A method for treating diabetes comprising administering to a subject in need of such a treatment, in addition to i) and ii),
   iii) allogeneic pancreatic islet cells or other insulin producing cells.
1.4 A method for inducing apoptosis of activated T cells in a subject in need of such treatment, comprising administering to said subject a therapeutically effective amount of a LFA-1 inhibitor in combination with at least one co-agent selected from a costimulation inhibitor and a mTOR inhibitor.
1.5 A method for delaying progression of, attenuating severity of, suppressing, mitigating or treating immune disorders or diseases in a subject by inducing or modulating immune tolerance in said subject, the immune disorders or diseases being dependent on activation of lymphoid cells, e.g. such as T cells, which method comprises administering to said subject a therapeutically effective amount of a LFA-1 inhibitor in combination with at least one co-agent selected from a costimulation inhibitor and a mTOR inhibitor.
   Examples of such disorders or diseases include e.g. rheumatoid arthritis, multiple sclerosis, systemic lupus erythematosus, dermatitis, and inflammatory bowel diseases, e.g. Crohn's disease or ulcerative colitis.
1.6 A method for treating malignancies in a subject in need thereof, comprising administering to said subject cells i) and a product ii) as indicated above, in order to achieve full or mixed hematopoietic chimerism.
   Examples of malignancies are e.g. hematologic malignancies, for example acute or chronic myeloid leukemias, multiple myelomas, non-Hodgkin's lymphoma, Hodgkin's disease, lymphomas etc., and other malignancies, e.g. breast cancer, testicular cancer, neuroblastomas, colon carcinoma etc.
1.7 A method for treating non-malignant diseases of bone marrow failure, comprising administering to said subject cells i) and a product ii) as indicated above, in order to achieve full or mixed hematopoietic chimerism.
   Examples of non-malignant diseases of bone marrow failure are e.g. aplastic anemia, thalassemia, sickle cell anemia, immunodeficiency disorders, Gaucher's disease, etc.

As alternative to the above the present invention also provides:
2. Use of a LFA-1 inhibitor in combination with at least one co-agent selected from a costimulation inhibitor and a mTOR inhibitor, e.g. In any of the methods 1.1 to 1.7 above, or in the manufacture of a medicament for use in such a combination, e.g. in any of the methods 1.1 to 1.7 above.
3. A pharmaceutical combination comprising
   a) a LFA-1 inhibitor; and
   b) at least one co-agent selected from a costimulation inhibitor and a mTOR inhibitor.
   A preferred combination is a LFA-1 inhibitor with a mTOR inhibitor or a LFA-1 inhibitor with a mTOR inhibitor and a costimulation inhibitor. Preferably the pharmaceutical combination of the invention is used in any of the methods 1.1 to 1.7 above.

By LFA-1 inhibitor is meant e.g. an agent that regulates ICAM-LFA-1 adhesion or LFA-1 induced costimulation and signaling, e.g. interrupts, modulates, or perturbs these functions of LFA-1. Preferably, the ICAM is ICAM-1, ICAM-2, or ICAM-3. Still more preferably, the ICAM is ICAM-1.

Agents that regulate ICAM-LFA-1 interaction encompass any chemical and/or biological molecules that do not prevent ICAM and LFA-1 from binding to each other, but nonetheless prevent a subsequent physiological effect of ICAM-LFA-1 binding. Additionally, agents that regulate ICAM-LFA-1 interaction encompass any chemical and/or biological molecules that prevent ICAM and LFA-1 from binding to each other, block binding, or inhibit binding. More particularly, the agents block ICAM-LFA-1 Interaction. Still more particularly, agents that regulate ICAM-LFA-1 interaction are polyclonal or monoclonal antibodies that bind to ICAM and/or LFA-1 and prevent ICAM and LFA-1 from binding to each other. Alternatively, agents that regulate ICAM-LFA-1 interaction are nonantibody compounds, such as small molecule antagonists, that bind to ICAM and/or LFA-1 and prevent ICAM and LFA-1 from binding to each other, or inhibit ICAM and/or LFA-1 expression.

Soluble ICAM-1 derivatives are also encompassed by the phrase “agents that regulate ICAM-LFA-1 interaction” Soluble ICAM-1 derivatives are derivatives which are not bound to a membrane of a cell. Such derivatives may comprise truncated molecules which lack a transmembrane domain. Alternatively, they may comprise mutant forms of the natural molecules which lack the capacity to be bound (or stably bound) to the membrane of a cell even though they contain a transmembrane domain. Soluble derivatives of ICAM-1 and their preparation are disclosed by Marlin, S. D. et al., Nature 344:70-72 (1990), which reference is incorporated herein by reference. Among the preferred functional derivatives of ICAM-1 are soluble fragments of the ICAM-1 molecule which contain domains 1, 2, and 3 of ICAM-1. More preferred are soluble fragments of the ICAM-1 molecule which contain domains 1 and 2 of ICAM-1. Most preferred are soluble fragments of the ICAM-1 molecule which contain domain 1 of ICAM-1. See U.S. Pat. No. 5,248,931.

Examples of LFA-1 small molecule antagonists include, but are not limited to, the molecules which bind e.g. to the so-called MIDAS-site (“metal ion dependent adhesion site”) or the L-site or south pole pocket (e.g. mevinolin or a derivative thereof, e.g. as disclosed in WO99/11258). Other examples are e.g.:
a) a compound as disclosed in WO 98/39303, e.g. a compound of formula: 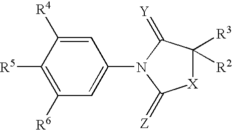
   wherein X, Y, Z and R² to R⁶ are as disclosed in WO98/39303, or a pharmaceutically acceptable salt thereof;
b) a compound as disclosed in WO01/30781, e.g. a compound of formula: 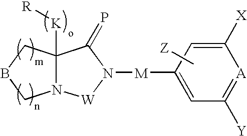
   wherein each group represented is as disclosed in WO01/30781, or a pharmaceutically acceptable salt thereof;
c) a compound as disclosed in WO01/92253, e.g. a compound of formula: 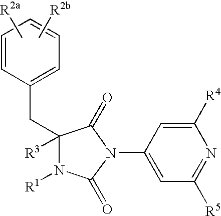
   wherein each group represented is as disclosed in WO01/92253, or a pharmaceutically acceptable salt thereof;
d) a compound as disclosed in WO01/07052, e.g. a compound of formula: 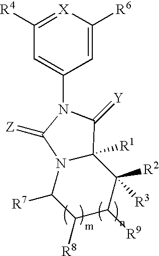
   wherein each group represented is as disclosed in WO01/07052, or a pharmaceutically acceptable salt thereof.

The contents of the publications referred to above are incorporated herein by reference.

Those of ordinary skill in the art can determine whether an agent regulates ICAM-1-LFA-1 interaction without undue experimentation. For example, an in vitro assay of ICAM-1-LFA-1 interaction is provided in U.S. Pat. No. 5,284,931 or in WO99/11258.

By costimulation is meant agonistic ligation of ligands and receptors involved in the regulation of antigen-receptor-induced function and/or activation and/or cell cycle progression. Such ligands and receptors include CD19, CD21, CD22, CD72, CD28, CD152, CD80, CD86, B7.h, LIGHT, ICOS, PD-1, PD-1 L, CD40, CD44, CD45, CD154, Ox40, Ox40L, CD137, CD137L. As targets for inhibition in synergy with LFA-1 inhibition, as stated in this invention, costimulation preferably includes positive costimulator ligand-receptor pairs of T lymphocyte activation such as Ox40-Ox40L, CD137-CD137L, CD28-CD80/CD86 and CD40-CD40L, even more preferably CD28-CD80/CD86 and CD40-CD40L.

By costimulation inhibitor is meant e.g. an agent that regulates, interrupts, modulates or perturbs costimulation ligand/receptor (e.g. CD40-CD154) interaction for use in the method of the invention. Agents that regulate such interaction encompass any chemical and/or biological molecules that do not prevent costimulation ligand-receptors from binding to each other, but nonetheless prevent a subsequent physiological effect of such binding. Additionally, agents that regulate costimulation ligand-receptor interaction encompass any chemical and/or biological molecules that prevent these molecules from binding to each other, block binding, or inhibit binding. Preferred costimulation inhibitors for use in the invention are e.g. agents that block CD40-CD154 interaction or CD28-CD80/CD86 interaction. More preferred agents that regulate costimulation ligand-receptor interactions are polyclonal or monoclonal antibodies, e.g. anti-CD154, or fusion proteins, e.g. CTLA4-lg or or a mutant thereof, that bind to such molecules and prevent them from binding to each other. Alternatively, agents that manipulate costimulation are non-antibody compounds, such as small molecule antagonists, e.g. that bind to CD40 and/or CD154 and prevent CD40 and CD154 from binding to each other.

Those of ordinary skill in the art can determine whether an agent regulates CD40-CD154 interaction without undue experimentation using in vitro assays. Such assays are disclosed, for example, in U.S. Pat. Nos. 5,683,693, 5,833,987, 5,869,049, 5,916,560, and WO97/26000.

“Anti-ICAM1 antibody” refers to an antibody that specifically recognizes and binds to an ICAM. Preferably, the antibody binds to ICAM-1, ICAM-2 or ICAM-3. Most preferably, the antibody binds to ICAM-1. Polyclonal and/or monoclonal antibodies can be used in the methods and combinations of the present invention. Anti-ICAM-1 antibodies can be made and used by those of ordinary skill in the art without undue experimentation. See U.S. Pat. No. 5,284,931. Examples of anti-ICAM-1 antibodies include, but are not limited to, monoclonal antibody R6-5-D6 (ATCC 9580), and YN1/1 (ATCC CRL-1878). A fragment of a complete anti-ICAM-1 antibody, which retains the activity of the complete anti-ICAM-1 antibody, is also suitable in the methods and combinations of the present invention. An active fragment of a complete anti-ICAM-1 antibody retains the activity of the complete antibody if the fragment regulates ICAM-1-LFA-1 interaction.

“Anti-LFA-1 antibody” refers to an antibody that specifically recognizes and binds to LFA-1. Pblyclonal and/or monoclonal antibodies can be used in the methods and compositions of the present invention. Anti-LFA-1 antibodies can be made and used by those of ordinary skill in the art without undue experimentation. For example, see U.S. Pat. No. 5,284,931. Examples of anti-LFA-1 antibodies include, e.g. monoclonal antibody M17/4.4 (ATCC TIB-217), TS2/18.1.1 (ATCC HB-195), TS1/22.1.1.13 (ATCC HB-202), TS1/18.1.2.11 (ATCC HB-203), LM2/1.6.11 (ATCC HB-204), TS2/9.1.4.3 (ATCC HB-205), 2E6 (ATCC HB-226), BE29G1 (ATCC HB-233), TS2/16.2.1 (ATCC HB-243), TS2/4.1.1 (ATCC HB-244), TS2/7.1.1 (ATCC HB-245), S6F1 (ATCC HB-9579), M5/114.15.2 (ATCC IIB-120), M1/70.15.11.5 HL (ATCC TIB-128), FD441.8 (ATCC TIB-213), M17/4.4.11.9 (ATCC TIB-217), M18/2.a.12.7 (ATCC TIB-218), M17/5.2 (ATCC TIB-237), and M5/49.4.1 (ATCC TIB-238).

A fragment of a complete anti-LFA-1 antibody, which retains the activity of the complete anti-LFA-1 antibody, is also suitable in the methods and combinations of the present invention.

An active fragment of complete anti-LFA-1 antibody retains the activity of the complete antibody if the fragment regulates ICAM-1-LFA-1 interaction.

“Anti-CD40 antibody” refers to an antibody that specifically recognizes and binds to CD40. Anti-CD40 antibodies can be made and used by those of ordinary skill in the art without undue experimentation. See U.S. Pat. No. 5,801,227. A fragment of a complete anti-CD40 antibody, which retains the activity of the complete anti-CD40 antibody, is also suitable in the methods and combinations of the present invention. An active fragment of a complete anti-CD40 antibody retains the activity of the complete antibody if the fragment interferes with CD154-CD40 interaction.

“Anti-CD154 antibody” refers to an antibody that specifically recognizes and binds to CD40 ligand. Anti-CD154 antibodies can be made and used by those of ordinary skill in the art without undue experimentation. See U.S. Pat. Nos. 5,683,693, 5,833,987, 5,869,049, 5,916,560 and WO97/26000. Examples of anti-CD154 antibodies include, but are not limited to, Genzyme (Cambridge, Mass.) anti-CD154 (product no. 80-3702-01), mouse anti-human CD154 antibodies 24-31, 89-79, 89-76, 24-43, 409-8 and 409-9, 5c8 (ATCC no. HB 10916), MR-1 (ATCC no. HB-11048), and BG9588 (see NIH Protocol Number: 99-AR-0133), anti-human CD154 MK13A4 (Pullen et al., J. Biol. Chem. (1999) from Alexis Biochemicals, or ABI793 (WO01/68860).

A fragment of a complete anti-CD154 antibody, which retains the activity of the complete anti-CD154 antibody, is also suitable in the methods and compositions of the present invention. An active fragment of a complete anti-CD154 antibody retains the activity of the complete antibody if the fragment regulates CD40-CD154 interaction.

The methods and compositions of the present invention can be made and practiced using any kind of suitable antibodies, including polyclonal, monoclonal, humanized or chimeric antibodies or fusion proteins.

A chimeric antibody is an antibody in which the light and/or heavy chains contain regions from different species. For example, one or more variable (V) region segments of one species may be joined to one or more constant (c) region segments of another species. Typically, a chimeric antibody contains variable region segments of a mouse joined to human constant region segments, although other mammalian species may be used.

A humanized antibody is an antibody comprising one or more complementarity determining regions (CDRs) of a non-human antibody functionally joined to human framework regions segments. Additional residues associated with the non-human antibody can optianally be present. Typically, at least one heavy chain or one light chain comprises non-human CDRs. Typically, the non-human CDRs are mouse CDRs.

The production of polyclonal, monoclonal, humanized or chimeric antibody is well-known to those of ordinary skill in the art. For example, see Antibodies: A Laboratory Manual, E. Harlow, Ed., Cold Spring Harbor Laboratory Press (1998); Vaswani, S. K. et al., Ann. Allergy, Asthma & Immunol. 81:105-115 (1998); Cuoto, R. et al., Cancer Res. (Suppl.) 55:5973s-5977s (1995); and U.S. Pat. No. 5,714,350.

“Active fragment” refers to a portion of a complete antibody that provides the same or improved therapeutic effect, as compared with the complete antibody.

By mTOR inhibitor as agent b) is meant a molecule that inhibits the function of mTOR such as, but not exclusively, rapamycin, i.e. sirolimus, (Abraham, R. T.: Wiederrecht G. J. Immunopharmacology of Rapamycin, Annu. Rev. Immunol. 1996, 14, 483-510) or a derivative thereof. Rapamycin is a known macrolide antibiotic produced by Streptomyces hygroscopicus, having the structure depicted in Formula A:

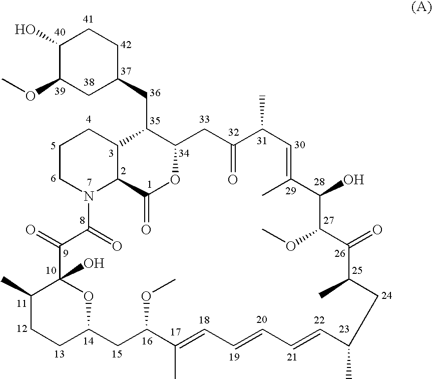

Of particular interest are rapamycin derivatives which are substituted in position 40, e.g. 40-O-substituted rapamycin derivatives as described in U.S. Pat. No. 5,258,389 and WO 94/09010, especially 40-O-alkylated rapamycin derivatives, e.g. wherein the 40-O-substituent is hydroxyalkylated, e.g. 40-O-(2-hydroxyethyl) rapamycin, i.e. everolimus, or derivatives substituted in position 40 and/or in other positions of the molecule, e.g. in position 28 and/or 16, including epimers thereof, and optionally further hydrogenated, e.g. as disclosed in WO 95/14023 and 99/15530, e.g. ABT578, TAFA-93, or rapalogs as disclosed e.g. in WO 98/02441 and WO01/14387, e.g. AP23573. 40-O-(2-hydroxyethyl) rapamycin is particularly preferred. Further preferred rapamycin derivatives are also compounds wherein the oxo in 32 is reduced, e.g. 32-deoxorapamycin or 16-pent-2-ynyloxy-32(S)-dihydro rapamycin.

According to the method of the invention, tolerance Is induced when the donor graft is accepted without the need for immunosuppressive treatments.

The above described method for inducing tolerance can be augmented by additional treatment regimens. For example, the method can further include partial whole body irradiation and/or local irradiation of the thymus gland before, at the same time, or after the exposure to the combined treatment.

Prior to the treatment of the invention, preferably prior to the administration of cells i), the recipient may also be submitted to a myelosuppressive therapy to facilitate hematopoietic stem cell engraftment, e.g. treatment with at least one compound selected from busulfan, fludarabine, 6-thioguanine, cyclophosphamide and T cell depleting antibodies such as anti-thymocyte globulin, anti-CD3 immunotoxin, Campath-1, etc., and/or submitted to irradiation.

The method of the invention can also include administering an immunosuppressant, immunomodulatory or costimulation blocking compound or a combination of such compounds before, at the same time or after the administration of agents a) and b). Examples of such compounds for adjunction include e.g. calcineurin inhibitor, e.g. cyclosporin A or FK 506; cyclophosphamide; azathioprene; methotrexate; leflunomide; mizoribine; mycophenolic acid or a salt thereof, e.g. Myfortic®; mycophenolate mofetil; 15-deoxyspergualine or an immunosuppressive homologue, analogue or derivative thereof; normal or high dose corticosteroid; an accelerating lymphocyte homing agent, e.g. FTY720; immunosuppressive monoclonal antibodies, e.g., monoclonal antibodies to leukocyte receptors, e.g., MHC, CD2, CD3, CD4, CD7, CD8, CD25, CD27, B7 (e.g. B7.1, B7.2 or B7.h), CD28, CD45, CD58, CD80, CD86, CD134, CD137, CD152, ICOS, 4-1BB, or OX40, or to their ligands, e.g. CD27-ligand, 4-1 BB-ligand, OX40-ligand; a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4lg (for ex. designated ATCC 68629) or a mutant thereof, e.g. LEA29Y.

A preferred combination comprises a) a LFA-1 inhibitor, b) a costimulation inhibitor and/or a mTOR inhibitor and c) 15-deoxyspergualine or an immunosuppressive homologue, analogue or derivative thereof.

The method of the invention can also be used with a patient that has undergone cell, tissue or organ transplant and is on an immunosuppressant regimen. This presents a significant opportunity to reduce or eliminate traditional immunosuppressant therapy and its well documented negative side-effects. Also treatment with immunosuppressants prior to transplantation could be particularly useful in cadaveric transplants.

Utility of the combination of agents a) and b) in tolerance induction, as well as in treating disease and conditions as hereinabove specified, may be demonstrated in animal tests for example in accordance with the methods hereinafter described, as well as in clinic where e.g. the transplanted organ, tissue or cell may be submitted to regular biopsy controls and in case of heart transplant additionally to ultrasound scanning.

Female 6-8 week old donor and recipient mice are used in a strain combination BALB/C (donor) to C57/BL6 (recipient) with a complete MHC mismatch.

Preparation of Bone Marrow

C57/BL6 donor mice are sacrificed by carbon dioxide inhalation and briefly placed in 70% ethanol. Muscles are dissected from femur, tibia and humeri, and the bones collected on ice in cold BMM (Media 199, Amimed, Switzerland) supplemented with 10 mM Hepes (Amresco, Solon, Ohio, U.S.A.), 10 μg/ml Dnase (Roche Diagnostic, Rotkranz, Switzerland), and 4 μg/ml gentamycin (Gibco BRL, Paisley, Scotland). The bone marrow (Bm) is flushed from the bones into a petri dish with BMM using a syringe and a 23 or 25 gauge needle for tibias and femurs or humeri, respectively. The Bm is repeatedly passed through a 19 gauge needle to make a single cell suspension. The suspension is passed through a 70 μM pore size nylon cell strainer (Falcon) and spun at 400 g for 5 min at 4° C. Bm cells are resuspended in cold BMM and the density of living cells, excluding red blood cells (ACK lysis in hemocytometer), adjusted to 20 million cells per 0.5 ml. Mice are injected i.v. into the tail vein with 0.5 ml cell suspension on day 0.

Preparation of Compounds and mAbs

Busulfan (Sigma, Switzerland) is dissolved in 20% DMSO 80% PEG/water, 50/50 at 3 mg/ml. Mice receive a single i.p. Injection of 30 mg/kg busulfan one day prior to BmTx. The mTOR inhibitor, e.g. 40-(2-hydroxyethyl)-rapamycin, is diluted in 20% KZI vehicle 80% PBS and injected i.p once daily from days 0-8 of BmTx at a dose of 3 mg/kg. The LFA-1 inhibitor and CD154 inhibitor used are Mabs which are protein G purified from hybridoma supernatants and diluted in PBS. Anti mouse CD154 mAb (clone MR-1) is injected i.p. at 0.5 mg per dose on days 0 and 4. Anti mouse LFA-1 (clone M17) is injected i.p. at 0.1 mg per dose on days 0, 2, and 4.

Evaluation of Hematopoietic Chimerism by Flow Cytometric Analysis.

The analysis of chimerism formation is designed to identify the following cell types: CD3+ CD4+ and CD3+ CD8+ T cells, B cells (B220+) and granulocytes (CD11 b+) are tested for the relative contribution of recipient and donor origin, based on allo MHC class I expression. The HD2d MHC class I molecule is expressed by donor but not recipient cells. Mice are briefly anaesthetized with isofluran and bled by punction of the vena saphena. Blood is collected in heparinized 1.5 ml conical tubes. For each staining 20 μl of blood are transferred to 1 ml-sized FACS tubes. All mABs and Streptavidin are purchased from Pharmingen/BD Biosciences, Switzerland. For Fc receptor blockade, 10 μl of purified rat anti mouse CD16/CD32 (Fcg III/II receptor) diluted 1/2000 in staining buffer (PBS, 2% BSA, 0.003% a-D-mannopyranoside, 0.7% EDTA, ph 6) is added to each tube and incubated for 30 min at RT on a CAT S20 shaker. For specific staining, two sets of 3 or 4-colour staining are set up for each blood sample. 10 μl of mAbs diluted and pre-mixed in staining buffer are added to tubes. For the first stain, mAbs include anti-CD3 FITC diluted 1/40, anti-H2Dd-biotin at 1/200, anti-CD8a-PerCP and anti-CD4-APC both diluted 1/40. The second stain includes anti-CD11b-FITC at 1/40, anti-H2Dd-biotin at 1/200, and anti-B220-PerCP at 1/40. Samples are incubated for 15 min at RT with gentle shaking. For washing, 200 μl ‘Cell Wash’ (optimized PBS supplied by BD Biosciences) are added to each tube and samples spun at 400 g for 5 min. Supernatants are aspirated, pellets resuspended and 20 μl of streptavidin-PE, diluted 1/10 in staining buffer, added to each tube for 15 min incubation at RT. For cell fixation and red blood cell lysis, 20 μl of CAL-Lyse (CALTAG, Burlingame, Calif., U.S.A.) are added for 10 min at RT followed by 500 μl destined water for another 5 min. Cells are spun at 400 g for 5 min and washed once with 500 μl ‘Cell Wash’. Cells are resuspended in 150 μl ‘Cell Wash’and analyzed on a FACS Calibur (BD Biosciences) using a protocol for 4 colour data acquisition. The instrument settings have been determined in a preliminary experiment using PBLs from BALB/C, C57/BL6 mice and mixed PBL samples with various combinations of antibodies and fluorescent-conjugated isotype control antibodies. Data are acquired and analyzed using CellQuest software. Further analysis and preparation of figures are performed in Microsoft Excel.

In order to study the kinetics of chimerism formation, PBL samples are stained and analyzed at various time points after BmTx, typically around days 14, 28, 56, and 86.

In above assays, synergy between anti-CD154 and anti-LFA-1 mAb treatment for chimerism induction is obtained (FIG. 2) whereas anti-LFA-1 alone had no effect. Furthermore, in a subsequent experiment, chimerism in the absence of anti-CD154 mAb is observed when anti-LFA-1 mAb is combined with 40-(2-hydroxyethyl)-rapamycin (FIG. 3). Overall levels of chimerism for each cell type are also obatined when applying any of these three synergistic principles, i.e. antiCD154+40-(2-hydroxyethyl)-rapamycin (FIG. 1), anti LFA-1+anti CD154 (FIG. 2 and FIG. 3), and LFA-1+40-(2-hydroxyethyl)-rapamycin (FIG. 3).

The cells i) and the combination of agents a) and b) may be administered to the recipient as separate entities either simultaneously, concurrently or sequentially with no specific time limits. When the LFA-1 inhibitor or the costimulation inhibitor is an antibody with a long half-life, it may be administered prior to the administration of the cells i). The cells i) and the combination of agents a) and b) may be administered to the recipient prior to transplantation of the organ, tissue or cells. For example, administration of the cells i) may be performed several days (e.g. 5 to 8 days) prior to cells, tissue or organ transplantation.

Administration of donor cells i) (in combination with the inhibitors) has been found to be sufficient for induction of tolerance to donor cells, tissue or organ. The number of cells i) administered may vary depending upon the type of cell used, the type of tissue or organ transplant, the weight of the recipient, the general condition of the recipient and other variables known to the skilled person. An appropriate number of cells i) for use in the method of the invention can be determined by a skilled person and will depend on the source of donor cells, pre-treatment of recipient and level of chimerism required for therapeutic benefit. Multiple injections of donor cells are within the scope by of this invention. Cells i) can be administered in a physiologically acceptable solution, such as a buffered saline solution or similar vehicle. Cells i) are preferably administered intravenously.

Agents a) and b) are administered in a biologically compatible form, optionally in association with one or more pharmaceutically acceptable diluent or carrier therefor. Suitable pharmaceutical compositions contain, for example, from about 0.1% to about 99.9%, preferably from about 1% to about 60%, of the active agent. The agents a) and b) may be administered separately at different times during the course of therapy or concurrently in divided or single combination forms.

The effective dosage of each agent employed in the method of the invention may vary depending on the particular compound or pharmaceutical composition employed, the mode of administration, the condition being treated, the severity of the condition being treated. The LFA-1 inhibitor or the costimulation inhibitor may be administered at a dose of from 0.1 mg to 2 g. It may be administered within a time-lag from −3 up to 6 months, e.g. every 3 or 6 days, until appropriate therapeutic levels of chimerism are achieved. A preferred dosage regimen comprises administration of 0.3 mg/kg to 5 mg/kg of LFA-1 antibody or 7 mg/kg to 40 mg/kg of CD154 antibody, starting on day −3 and with an interval of 2 days upt to day 30 and subsequently once weekly up to 6 months. The antibody may conveniently be administered parenterally or intraperitoneally, e.g. intravenously. Smaller doses and/or less frequent dosing may be preferred.

Daily dosages for the mTOR inhibitor will, of course, vary depending on a variety of factors as indicated above. In general, however, satisfactory results are achieved on administration of rapamycin or a derivative thereof at daily dosage rates of the order of ca. 0.25 to 25 mg, particularly 0.75 to 25 mg, as a single dose or in divided doses. The mTOR inhibitor may conveniently be administered enterally, e.g. orally.

In a preferred embodiment, a LFA-1 inhibitor and/or a co-receptor inhibitor and/or a mTOR inhibitor will be administered 1-3 days before administration of bone marrow or precursor cells and up to a time of six months after hematopoietic chimerism has been established.

Preferably agent a) is a LFA-1 antibody. Preferred CD154 inhibitor as agent b) is an anti-CD154 antibody, preferably a humanized or human anti-CD154 antibody. Preferred mTOR inhibitor as agent b) is 40-O-(2-hydroxyethyl)-rapamycin. Preferred combinations are those comprising an LFA-1 inhibitor, e.g. a LFA-1 antibody, and 40-O-(2-hydroxyethyl)-rapamycin; or a combination comprising a LFA-1 inhibitor, e.g. a LFA-1 antibody and a CD154 inhibitor, e.g. an anti-CD154 antibody; or a combination comprising comprising an LFA-1 inhibitor, e.g. a LFA-1 antibody, 40-O-(2-hydroxyethyl)-rapamycin and a CD154 inhibitor, e.g. an anti-CD154 antibody.

1. A method for inducing or modulating T or B cell tolerance to donor cells, tissue or organ in a recipient comprising administering to the recipient a LFA-1 inhibitor in combination with a costimulation inhibitor and/or a mTOR inhibitor.

2. A method for inducing hematopoietic chimerism in a recipient of cells, tissue or organ transplant from a donor comprising administering to the recipient
i) bone marrow cells or other precursor cells from the donor; and
ii) a LFA-1 inhibitor in combination with at least one co-agent selected from a costimulation inhibitor and a mTOR inhibitor.

3. A method for treating diabetes comprising administering to a subject in need of such a treatment, in addition to i) and ii) as defined in claim 2,
iii) allogeneic pancreatic islet cells or other insulin producing cells.

4. A method for inducing apoptosis of activated T cells in a subject in need of such treatment, comprising administering to said subject a therapeutically effective amount of a LFA-1 inhibitor in combination with at least one co-agent selected from a costimulation inhibitor and a mTOR inhibitor.

5. A method for delaying progression of, attenuating severity of, suppressing, mitigating or treating immune disorders or diseases in a subject by inducing or modulating immune tolerance in said subject, the immune disorders or diseases being dependent on activation of lymphoid cells, which method comprises administering to said subject a therapeutically effective amount of a LFA-1 inhibitor in combination with at least one co-agent selected from a costimulation inhibitor and a mTOR inhibitor.

6. A method for treating malignancies in a subject in need thereof, comprising administering to said subject cells i) and a product ii) as defined in claim 2, in order to achieve full or mixed hematopoietic chimerism.

7. A method for treating non-malignant diseases of bone marrow failure, comprising administering to said subject cells i) and a product ii) as defined in claim 2, in order to achieve full or mixed hematopoietic chimerism.

8. A method according to claim 1, further comprising administering to the recipient or subject 15-deoxyspergualine or an immunosuppressive homologue, analogue or derivative thereof.

9. Use of a LFA-1 inhibitor in combination with at least one co-agent selected from a costimulation inhibitor and a mTOR inhibitor in a method according to claim 1.

10. A pharmaceutical combination comprising
a) a LFA-1 inhibitor; and
b) at least one co-agent selected from a costimulation inhibitor and a mTOR inhibitor.

11. A combination comprising
a) a LFA-1 inhibitor; and
b) at least one co-agent selected from a costimulation inhibitor and a mTOR inhibitor for use in a method according to claim 1.

12. A combination according to claim 10, further comprising 15-deoxyspergualine or an immunosuppressive homologue, analogue or derivative thereof.
